(19) United States
(12) Patent Application Publication
(10) Pub. No.: US 2003/0158603 A1
(43) Pub. Date: Aug. 21, 2003

(54) Apparatus and method for harvesting bone

(75) Inventor: Peter R. Ebner, Hollis, NH (US)

Correspondence Address: Norman P. Soloway, HAYES SOLOWAY P.C, 130 W. Cushing Street, Tucson, AZ, 85701, US

(73) Assignee: MAXILON LABORATORIES, INC.

(21) Appl. No.: 10/080,193

(22) Filed: Feb. 19, 2002

(51) Int. Cl. A61F 2/28

(52) U.S. Cl. 623/16.11

(57) ABSTRACT

A bone grafting and/or shaping instrument includes a blade assembly formed from a section of tubing and shaped to form a truncated cone.

### FIELD OF THE INVENTION

The present invention relates to the field of surgery. The invention has particular utility in connection with the removal and collection of bone from the surface of one or more donor sites, and the preparation and placement of the autogenous bone material at a second location in the patient, e.g. for use in grafting bone to osseous deficiencies, such as periodontal and dentoalveolar defects, bone deficiencies around dental implants, and numerous orthopedic applications that require grafting.

### BACKGROUND OF THE INVENTION

Many reconstructive procedures used in medicine and dentistry involve the manipulation and healing of bones. Such procedures may involve changes in the position, orientation, shape and size of skeletal structures. A problem that is commonly encountered during such procedures is a lack of bone graft material. Bone graft material may be used in several applications, such as to fill between sections of bone that have been repositioned, to change surface geometry, or to add bone to an area that is deficient, such as in conjunction with periodontal surgery or dental implants in the patients' jaws.

The need to harvest small bone grafts from intraoral sites has been common in periodontal surgery to restore bone defects around teeth. In the case of dental implant surgery, bone grafts may be needed to augment atrophic alveolar ridges of the maxilla and/or mandible and the sinus floor to increase the dimension of these bone sites to accommodate and totally cover the endosseous portion of implant fixtures. Bone grafts also are used in conjunction with guided tissue regeneration; a technique that uses a membrane to isolate hard tissue from soft tissue sites and potentiates hard tissue healing.

It is often difficult to harvest adequate amounts of autogenous bone from intraoral sites. Therefore, clinicians often rely on non-autogenous sources of graft material, such as bone from cadaver sources (homologous or allogenic grafts), animal sources (heterogenous or xenogeneic grafts), or synthetic bone substitutes. However, healing of non-autogenous material grafts is not as extensive or predictable as healing of autogenous bone obtained directly from the patient; plus there is the additional cost of such nonautogenous graft materials which can be significant.

Clinicians use several techniques to remove bone for grafting for intraoral procedures. In one such technique rotary instruments, such as side cutting burrs or trephines, are used to remove a piece or section of cortical bone from a local intraoral site in the maxilla or mandible. The cortical bone is often morsalized into a particulate form, either manually with a rongeur like instrument or in a bone mill. The particulate bone is then combined with blood to form an osseous coagulum, which is then positioned and packed into the osseous defect around the teeth or implant. See Robinson, R. E. “Osseous Coagulum for Bone Induction”, J. Periodontology 40:503(1969). Suction devices with filters have been fabricated and manufactured to collect the bone dust from rotary instruments. See Hutchinson, R A “Utilization of an Osseous Coagulum Collection Filter”, J. Periodontology 44:668(1973). See also Goldman, et al., “Periodontal Therapy”, pp 994-1005, C. V. Mosby Co., (1980); and Haggarty, et al., “Autogenous Bone Grafts: A Revolution in the Treatment of Vertical Bone Defects”, J. Periodontology 42:626(1971). While such techniques are widely used by clinicians, the techniques have limitations, since sites to harvest sections of intraoral bone are limited in number and extent because of limited intraoral access, proximity to tooth roots, nerve structures and sinus cavities, and thin plates of bone.

Other techniques for harvesting bone include using chisels or osteotomes to remove and manually collect shavings from the surface. These instruments must be very sharp and the process is often awkward and time consuming. Other manual instruments such as bone files and rasps also remove bone. However, the efficiency of cutting and the ability to use the removed bone is greatly limited. Another technique is to collect bone dust generated by twist drills or taps used to prepare the sites for implant placement. However, much of the bone material may be lost while the site is being irrigated to cool the cutting instrument. When larger amounts of bone are needed for major reconstructive procedures, other sites such as the hip (anterior or posterior ilium), tibia, ribs, or the calvarium often are used. However, using such other sites necessitates a second surgical site, which may require postoperative hospitalization, and thus is less amenable, e.g. in the case of an outpatient dental procedure.

Various surgical devices have been proposed and/or are in use to harvest bone marrow samples for biopsy or devices such as rongeurs or bone cutters or punches to remove sections or convex edges of bone. Surgical devices also are in use in arthroscopy and endoscopy for cutting or drilling bone or tissue and removing the tissue fragments. Ultrasonic devices to cut bone also are in use; however, such devices require the removal of the irrigant and debris liberated by the apparatus. Each of these methods and/or devices, however, suffers from one or more deficiencies as applied to the collection of bone for grafting.

Yet other patented devices have been proposed; each of these, however, suffers from one or more deficiencies:

U.S. Pat. Nos. 5,403,317 and 5,269,785 to Bonutti show a method and apparatus for the percutaneous cutting and removal of tissue fragments from human. The Bonutti device removes the tissue fragments by suction. Wherein it can be collected and then placed elsewhere in the patient from where originally obtained. Bonutti employs a flexible drill, and suction to remove the debris to an externally placed collection reservoir, where it is compressed before being replaced into the patient.

U.S. Pat. No. 2,526,662 to Hipps discloses a bone meal extractor apparatus for mechanically removing bone meal from a donor bone site through a small percutaneous site using a drill. The drill shavings, which comprise primarily sub-surface bone, are then evacuated into an open cut that the drill passes through, for collection.

U.S. Pat. No. 4,798,213 to Doppelt teaches a device for obtaining a bone biopsy for diagnosis of various bone diseases. The Doppelt device is intended to remove a core of bone using a tubular drill, while maintaining the architecture of the tissue. The sample is obtained from the marrow space and not intended from re-implantation.

U.S. Pat. No. 5,133,359 to Kedem shows a hard tissue biopsy instrument in which samples are taken using a rotatably driven hollow needle.

U.S. Pat. No. 4,366,822 to Altshuler discloses a method and apparatus for bone marrow cell separation and analysis. The Altshuler apparatus collects bone marrow cells in a filtration chamber on a filter interposed between a needle directed into the bone marrow site and an aspirator or vacuum source, i.e. using negative pressure to withdrawal marrow cells through a needle.

U.S. Pat. No. 5,052,411 to Schoolman teaches, a vacuum barrier attachment for shielding the operator of a medical tool from harmful aerosols and blood, etc. created by drilling, sawing types of actions, etc. The Schoolman device requires vacuum and is not intended for harvesting tissue for re-implantation.

U.S. Pat. No. 4,722,338 to Wright et al. discloses a device instrument for removing bone which uses a shearing action similar to a rongeur to cut bone, with means for collecting fragments of bone as they are removed. The Wright et al. device reportedly is used mainly for the removal of projections or edges of bone using a shearing mechanism without the intent of harvesting the bone for grafting.

U.S. Pat. No. 4,994,024 to Falk teaches an arthroscopy hook-clippers device that allows the unobstructed removal of tissue or bone with removal of the fragments by suction. The Falk device is intended for arthroscopy applications and with the removal of projections of tissue or bone and not specifically for the harvest of tissue for grafting.

Yet other prior art devices are disclosed in U.S. Pat. No. 4,466,429 to Loscher et al. and U.S. Pat. No. 4,844,064 to Thimsen et al.

The foregoing discussion of the prior art derives from my earlier PCT Application No. WO 97/11646, which describes a hand-held surgical instrument for the cutting, removal, and storage of bone surface shavings for use as autogenous bone grafts. The instrument is comprised of a blade mounted in a handle for holding and supporting said blade. The blade has a cutting structure adjacent its distal end in the form of a sharpened loop. The loop's wedge shaped cross-section is defined distally by a perpendicular curved aperture through the blade, and distally by a ground and honed relief. In the preferred form, the handle cooperates to provide a storage space adjacent the distal end of the blade for receiving harvested bone from the cutting structure. This manual instrument is held at an acute angle to the bone, and with minimal downward pressure, is drawn across the bone surface to cut and collect a thin shaving of bone. The blade is preferably retractable to allow the clinician access to the harvested material. A plunger is incorporated into the handle to serve both as a locking mechanism to secure the blade and as a means to advance and consolidate the bone in the distal aspect of the instrument.

The present invention provides substantial improvements, enhanced functionality and reduced cost, over the surgical instrument described in my aforesaid PCT Application No. WO 97/11646.

### SUMMARY OF THE INVENTION

The invention is directed to a hand-held surgical instrument for the cutting, removal, and storage of bone surface shavings for use as autogenous bone grafts. The instrument is comprised of a blade assembly mounted to a handle for holding and supporting the blade assembly. The ring-shaped blade has a cutting surface made from a section of metal tubing such as 440A stainless steel tubing, or ceramic tubing, or other hard material such as monocrystalline sapphire, that is oriented relative to a longitudinal axis of the handle to allow the operator to more easily cut or scrape and accumulate bone from hard-to-reach locations. The blade assembly preferably includes a flexible region that allows a single blade design to be inserted into a variety of handles having different nose angles. Several different handles allow the surgeon to select a handle that orients the blade at a desired angle relative to the longitudinal axis of the handle. In some embodiments of the present invention, the ring-shaped blade may be fit to its carrier so as to allow it to be rotated about its central axis. This enables the clinician to move, with a pair of needle holders or similar instrument if necessary, an unused portion of the cutting edge into the cutting position and thereby extend the usefulness of the blade. The handle optionally may include a storage space adjacent the distal end of the instrument for receiving harvested bone from the blade. The handle may be enlarged at the proximal end to provide a bowl like mixing area that communicates with the storage space. This enlargement may also serve as an improved grip to reduce operator fatigue. In use, the instrument is held at an acute angle to the bone, and with minimal downward pressure, drawn across the bone surface to cut and collect a thin shaving of bone. The blade preferably is retractable to allow the clinician to access and deliver the harvested material. A plunger may be incorporated into the handle to serve both as a locking mechanism to secure the blade and as a means to advance and consolidate the bone in the distal end of the instrument. In a preferred embodiment, the blade is removable and replaceable, while the handle and the plunger are reusable.

In another preferred embodiment of the invention, the handle has an area of reduced mechanical strength or a flexible joint displaced from the cutting blade, the area of reduced mechanical strength allowing the cutting blade cutting edge to be angularly positioned relative to a longitudinal axis of the blade assembly.

The above and other objects, features, and advantages of the present invention will be apparent in the following detailed description thereof when read in conjunction with the appended drawings wherein the same reference numerals denote the same or similar parts, and wherein:

### BRIEF DESCRIPTION OF THE DRAWINGS

FIG. 1 is a perspective view of a first exemplary bone-harvesting instrument consistent with the present invention;

FIG. 2 is an enlarged view of a portion of the instrument of FIG. 1;

FIG. 3 is a bottom plan view of a portion of the instrument of FIG. 1;

FIG. 4 is a section view of a portion of the instrument in FIG. 1 taken through a line 4-4 in FIG. 3;

FIG. 5 is a side elevational view of a portion of the instrument in FIG. 3;

FG. 6 is a side elevational view of a portion of the instrument of FIG. 1, showing the instrument in flexure;

FIG. 7 is an exploded view of a second exemplary bone-harvesting instrument consistent with the present invention;

FIG. 8 is a view, similar to FIG. 3, of a portion of the instrument of FIG. 7;

FIG. 9 is an end view of the instrument of FIG. 8 taken through a line 9-9 in FIG. 8;

FIG. 10 is a plan view illustrating manufacture of a cutting blade from tubing stock; and

FIG. 11 is an enlarged view showing details of a portion of FIG. 10.

### DETAILED DESCRIPTION OF THE INVENTION

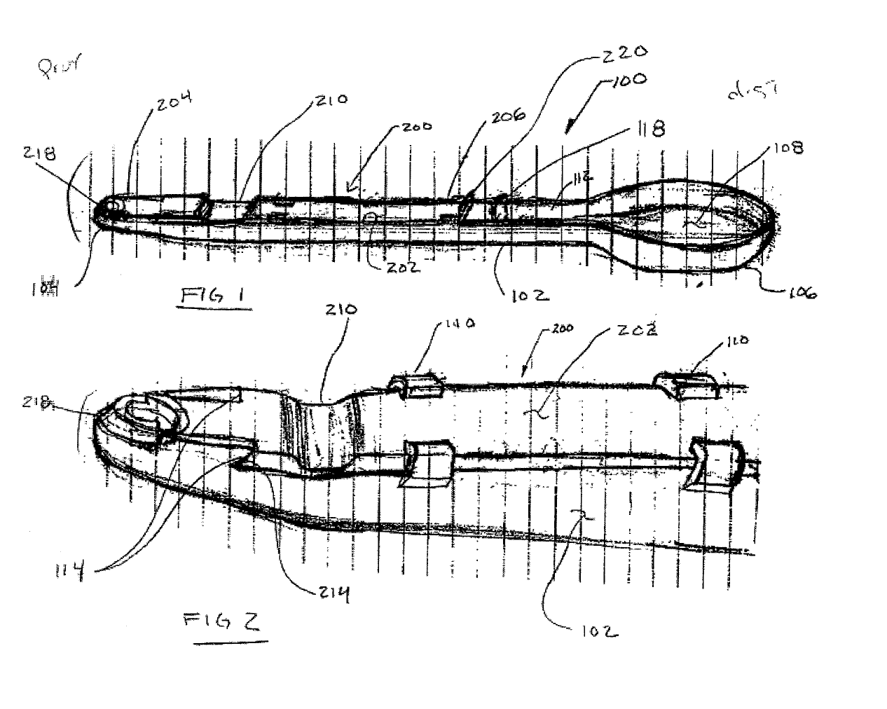

The general arrangement of the elements is shown most clearly in FIG. 1 and FIG. 7. FIG. 1 shows the bone-harvesting instrument 100 including an elongated body 102 and, a blade assembly 200, and a plunger 118. The body 102 serves as a handle for the instrument 100 and supports the blade assembly 200. The body 102 has a distal end 104 and proximal end 106, and a channel 112 that extends from the distal end 104 to a mixing area 108 disposed in proximity to the proximal end 106. The blade assembly 200 and the elongated body 102 cooperate to form the channel 112. The mixing area 108 is used to mix shavings with blood and other materials as will be described below to make a composition that can be later applied to an area of a patient needing an autogenous bone graft. The plunger 118 is slidable and is used to urge the composition from the mixing area 108 through the channel 112 and to the distal end 104. The channel 112 preferably is shaped to increase in cross-sectional area, except in plunger travel section, from the distal end 104 to the proximal end 106, so as to help urge the bone fragments towards the mixing area 108. The elongated body 102 and the plunger 118 may be made from metal, such as stainless steel, or a medically approved plastic such as Acrylis Cyro GS90.

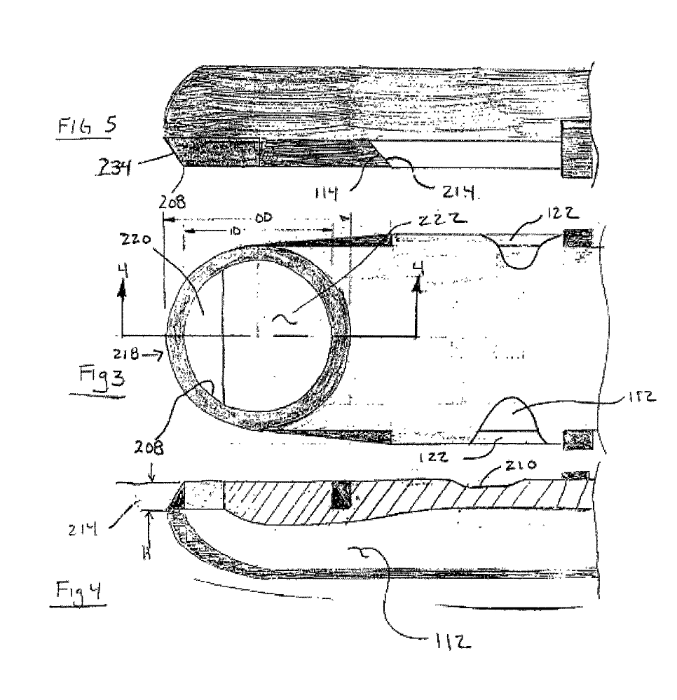

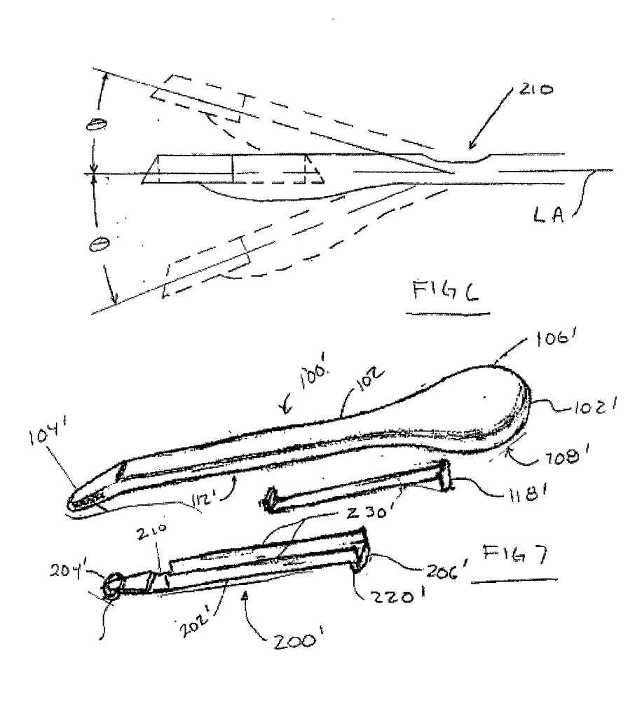

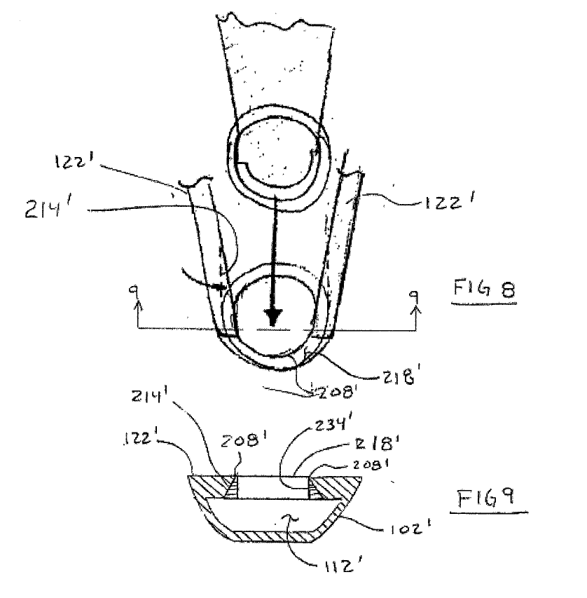

The blade assembly 200 includes a blade carrier 202, an elongate generally flat member, a blade 218 adjacent a first end 204 and a tab 220 at a second end 206. Tab 220 enables the user to slide the blade assembly and acts to interrupt travel of the blade assembly and the plunger. A feature and advantage of the present invention is to provide a low cost blade that is simple to manufacture. Thus, in a preferred embodiment, the blade 218 is formed from a section of tubing having a height “H”, an inside dimension “ID” and an outside dimension “OD” (see FIG. 3). The tubing is shown as a hollow cylinder, but other shapes will work. The blade may be formed by cutting suitably dimensioned tubing at an angle. Alternatively, the tubing may be cut into short stub-sections, and the sections ground into a truncated cone shape to form a cutting surface 208. The blade 218 is secured to the blade carrier 202 by press fitting the tubing onto a protrusion 222. The press fit, and/or an under cut, can be dimensioned to allow the clinician to rotate the ring-shaped blade about its central axis enabling unused portions of the cutting edge to be moved into the active position. The protrusion 222 may be circular with a portion removed leaving an opening 220 so that bone scraped by the cutting surface 208 may enter the channel 112 through the opening 220. Alternatively, the blade 218 may be secured to the blade carrier 202 using an adhesive, a mechanical fastener, or the blade may be fixed in grooves 214′ (see FIG. 8) as will be discussed below. The blade 218 may be metallic, preferably 440A stainless steel, or a ceramic, or other hard material such as monocrystalline sapphire. The blade carrier 202 may be made from plastic or the same material as the cutting surface, i.e., metal or ceramic, or a different material. Preferably, the blade carrier 202 is formed of a transparent or translucent plastic such as Acrylis Cyro GS90, so as to provide visual feedback of the accumulated bone material to the user.

The blade assembly 200 rests on sidewalls 122 of the body 102, and is secured in place along the sidewalls by retainers 110 and near the distal end 104 by an undercut 114. The undercut 114 on the body 102 cooperates with a protrusion 214 on the blade carrier 202 to help maintain the distal end 204 of the blade assembly 200 in contact with the distal end 104 of the handle 102. The width of the handle 102 along the longitudinal axis LA may be reduced near the end 104 of the handle 102. Alternatively, as described later with respect to FIGS. 7-9, the blade assembly may be secured in a pair of grooves formed in an inside surface of the body 102, the grooves extending along the longitudinal axis of the body 102.

As shown in FIG. 6, in a preferred embodiment of the invention, the blade assembly 200 includes an area of reduced mechanical strength 210 in the blade carrier 202 that is displaced from the blade 218. The area of reduced mechanical strength 210 may be an area where the thickness is less than the thickness of a surrounding area. Alternatively, the area of reduced mechanical strength may be an area where the material has a lower modulus of elasticity than the surrounding area. The area of reduced mechanical strength 210 allows the blade carrier 202 to flex. This allows the cutting surface 208 of the blade 218 to be positioned at a desired angle relative to the longitudinal axis of the body 102. The body 102 may be contoured such that when the blade assembly is secured to the body, the cutting surface is at an angle relative to the longitudinal axis of the body 102. The angle θ may range between +/−45°, preferably less than 30° and more preferably less than 15°.

FIGS. 7-9 show a second exemplary bone-harvesting instrument 100′ including an elongated body 102′, a blade assembly 200′, and a plunger 118′. The body 102′ serves as a handle for the instrument 100′ and supports the blade assembly 200′. The body 102′ includes a distal end 104′ and proximal end 106′. The body 102′ has a channel 112′ that extends from the distal end 104′ to a mixing area 108′ disposed in proximity to the proximal end 106. The blade assembly 200′ includes a blade carrier 202′ with downwardly extending wall portions 230′ that fit within sidewalls 122′ of the elongated body 102′. The extending wall portions 230′ provide a friction fit that retains the blade assembly 200 in the elongated body 102 and add structural strength to the member 202′. The blade assembly 200′ and the elongated body 102′ cooperate to form the channel 112. The mixing area 108′ is used to mix shavings of scraped bone and blood with other materials such as xenogeneic bone, allogenic bone, alloplastic material (hydroxyapatite), platelet rich plasma, and/or recombinant growth factors (BMP) to make a composition that can be later applied to an area of a patient needing a bone graft. The mixing area is at the proximal end to also serve as an improved grip.

The blade 218′ may be formed from a section of tubing similar to the blade shown in FIG. 1, and may be secured to the member 202′ the same way as shown in FIG. 1. The member 202′ may be formed of the same material as the blade 218′ or a different material. Preferably, the member 202′ is formed of a transparent or translucent plastic.

The blade assembly 200′ is secured in place along the elongated sides by friction and/or retainers (not shown). The sidewalls 122′ near the end 104′ of the elongated body 102′ includes an undercut 214′ shaped such that when the blade 218′ is inserted, the sidewalls 122′ of the body 102′ contact the outside wall surface 234′ of the blade 218′ and resist movement of the blade 218′. The undercut resists movement of the blade 218′ in a direction perpendicular to the longitudinal axis or along the longitudinal axis of the body 102′. The blade assembly 200′ also may include an area of reduced mechanical strength 210′ in the member 202′ that is displaced from the blade 218′.

The body 102′ is contoured such that when the blade assembly 200 is secured to the body, the cutting surface is at a desired angle relative to the longitudinal axis LA of the body 102′. The angle θ may range between +/−45°, preferably less than 30° and more preferably less than 15°.

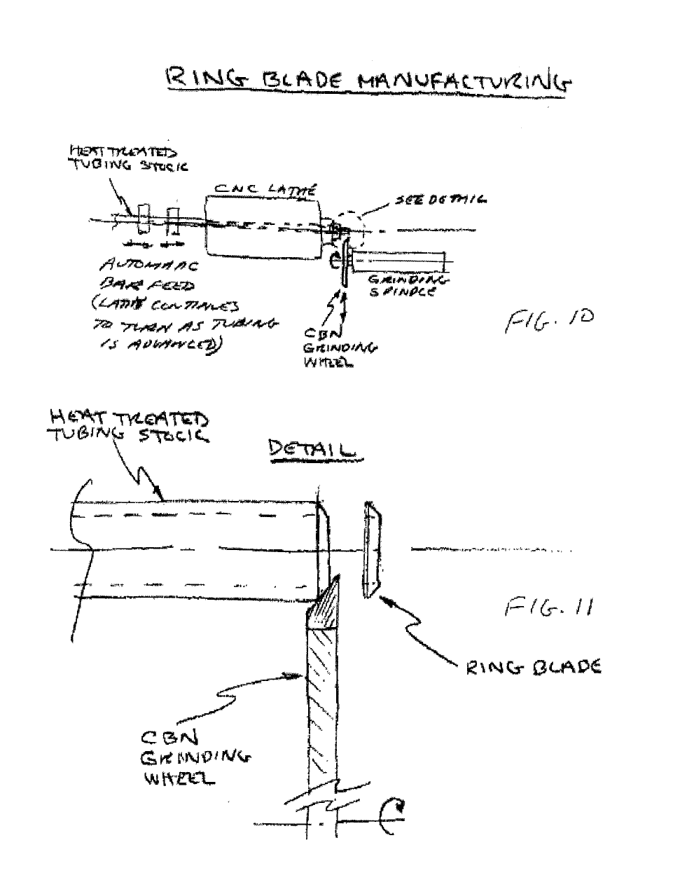

The present invention provides several significant advantages. For one, forming the cutting blades from heat-treated, hardened and tempered tubing simplifies manufacturing and enables very efficient use of raw material since a minimal amount of material is contained in the finished blade because of its geometry. Moreover, as shown in FIGS. 10 and 11, the finished cutting blades can be made on a standard CNC lathe with automatic bar feed. This has the benefit of low tooling cost and high production rate capability. Furthermore, because minimal material needs to be removed, blades can be made by simple grinding of heat-treated tubing such as 440A stainless steel tubing. This eliminates individual handling, heat-treating and sharpening of each blade.

The ring shape of the blade also enables it to be directly coupled to the handle with the use of slots that terminate in ends that match the shape of the blade. This allows the high cutting force to be transferred directly to the blade, while enabling it to be easily inserted and removed from the handle. Because the blade carrier is not subjected to high cutting force, it can be made of relatively thin transparent plastic that serves as a window for the graft material as it is being harvested. Also, because the blade carrier can be made transparent, the handle can be made of metal. Metal has sufficient strength, can be autoclaved, and is suitably durable for re-use. Yet another advantage of the use of a ringshaped blade is that the blade can be rotated to position a new portion of the cutting edge at the cutting location.

Yet other features and advantages of the present invention are realized by the use of a flexible blade carrier which enables a standard blade assembly to be used in handles with various nose angles. Alternatively, the flexible blade carrier may be made to conform to changes in angle when used with an adjustable nose angle handle.

Other features and advantages of the present invention derive from the enlarged proximal end of the handle. This provides a mixing bowl integrated into the grafting instrument combined with an improved grip that reduces user hand fatigue. Additionally, the enlarged proximal end of the handle communicates with the collection chamber enabling harvested graft material to be pushed into the bowl area for mixing with other materials such as, for example, a volume expander such as bio-oss or recombinant growth factors such as BMP. After mixing, the mixture can be pushed back into the collection area for delivery to the recipient's site.

It should be understood that, while the present invention has been described in detail herein, the invention can be embodied otherwise without departing from the principles thereof, and such other embodiments are meant to come within the scope of the present invention as defined in the following claims.

1. A surgical instrument for bone grafting and/or shaping bone, comprising:
a blade assembly having a cutting surface and an area of reduced mechanical strength displaced from the cutting surface, the area of reduced mechanical strength allowing the cutting surface to be angularly positioned relative to a longitudinal axis of the blade assembly.

2. The instrument of claim 1, further comprising an elongated handle having a distal end and a proximal end, the handle coupleable to the blade assembly.

3. The instrument of claim 2, wherein the handle comprises a mixing area disposed at the proximal end of the handle for mixing the bone, blood and other constituent graft materials.

4. The instrument of claim 2, wherein the handle further comprises a protrusion for mounting the blade assembly on the handle.

5. The instrument of claim 2, wherein the handle further comprises a channel or groove for mounting the blade assembly on the handle.

6. The instrument of claim 2, further comprising a plunger for moving material within the instrument.

7. The instrument of claim 1, wherein the cutting surface is formed on a section of a blade of ring-like shape.

8. The instrument of claim 7, wherein the blade comprises a section of metal, ceramic or other hard material tubing.

9. The instrument of claim 1, wherein the area of reduced mechanical strength comprises a flexible joint.

10. The instrument of claim 1, wherein the cutting surface is coupled to the area of reduced mechanical strength by a body portion.

11. The instrument of claim 10, wherein the body portion comprises a plastic material.

12. The instrument of claim 10, wherein the cutting surface and the body portion comprise different materials.

13. The instrument of claim 10, wherein the body portion comprises a transparent or translucent plastic material.

14. The instrument of claim 2, wherein the cutting surface is positioned substantially parallel to the longitudinal axis of the handle.

15. The instrument of claim 2, wherein the cutting surface is positioned at an angle relative to the longitudinal axis of the handle.

16. The instrument of claim 10, wherein the cutting surface is coupled to the body portion by compression fit, adhesive, or an interlocking arrangement.

17. The instrument of claim 2, wherein the blade assembly includes a collection chamber sized and shaped to fit within said handle.

18. An instrument for bone grafting and/or shaping bone, comprising:
an elongate body having a proximal end and a distal end; the body serving as a handle for the instrument, and supporting a cutting blade of a ring-like shape.

19. The instrument of claim 18, wherein the body includes a storage compartment, and further comprising a plunger for moving graft material within the instrument.

20. The instrument of claim 18, wherein the cutting blade is formed from a section of tubing.

21. The instrument of claim 20, wherein the tubing comprises metal, ceramic or other hard material.

22. The instrument of claim 21, wherein the tubing comprises monocrystalline sapphire tubing or stainless steel tubing.

23. The instrument of claim 19, wherein the storage compartment comprises a disposable channel shaped component.

24. An instrument for bone grafting and/or shaping bone, comprising:
an elongate body having a distal end and distal end; the body serving as a handle for the instrument, and supporting a blade assembly therein;
the blade assembly having a cutting surface and an area of reduced mechanical strength displaced from the cutting surface, the area of reduced mechanical strength allowing the cutting surface to be angularly positioned relative to a longitudinal axis of the blade assembly.

25. The instrument of claim 24, wherein the cutting surface is formed on a truncated, hollow cone, the side wall of the truncated cone cooperating with an undercut portion on the elongated body to maintain the truncated cone of the blade assembly in contact with the elongated body.

26. The instrument of claim 25, wherein a protrusion on the blade assembly cooperates with an undercut portion on the elongated body to maintain the blade assembly in contact with the elongated body.

27. An instrument for bone grafting and/or shaping bone, comprising:
an elongate handle having a proximal end and a distal end, and supporting a blade assembly therein;
the blade assembly having a first end adjacent the distal end of the handle and separated from a proximal end by a body portion, the body portion having a longitudinal axis, the blade assembly coupleable to the handle to reduce the flexibility of the blade assembly, the distal end of the blade assembly having a cutting surface, the body portion having an area of reduced mechanical strength disposed between the distal end and the proximal end to allow the cutting surface to be oriented at a plurality of angles relative to the longitudinal axis.

28. The instrument of claim 27, further comprising a plunger for moving graft material within the instrument.

29. The instrument of claim 27, wherein the handle comprises a hollow having a longitudinal opening covered by said blade assembly which creates a central storage space within the handle, and the plunger provides moveable access to the storage space.

30. The instrument of claim 27, further comprising a retainer for coupling the blade assembly to the handle.

31. The instrument in claim 27, wherein the handle is of an ergonomic streamline shape, with a shallow distal profile for accessing greatly narrowed spaces.

32. The instrument of claim 27, wherein a portion of the blade assembly is comprised of a transparent or translucent plastic material.

33. The instrument of claim 27, wherein the handle is comprised of a transparent or translucent plastic material.

34. The instrument of claim 32, further comprising indicia on the blade assembly for providing an indication of the amount of collected bone material.

35. The instrument of claim 29, wherein the channel in the handle expands in cross-sectional area along a part of its length from the distal end to the proximal end.

36. The instrument of claim 29, wherein the blade assembly includes a collection chamber sized and shaped to fit within the channel.

37. The instrument of claim 7, wherein the blade is rotatably mounted in the instrument so as to permit the exposure of fresh cutting surfaces.

38. The instrument of claim 37, wherein the blade is rotatably mounted in grooves formed on the inside wall of said instrument.

39. The instrument of claim 3, wherein the handle comprises an enlarged proximal end for accommodating said mixing area and for providing a grip.

40. The instrument of claim 18, wherein the handle comprises an enlarged proximal end for accommodating a mixing area and for providing a grip.

41. The instrument of claim 24, wherein the handle comprises an enlarged proximal end for accommodating a mixing area and for providing a grip.

42. The instrument of claim 27, wherein the handle comprises an enlarged proximal end for accommodating a mixing area and for providing a grip.

43. The instrument of claim 18, wherein the blade is rotatably mounted in the instrument so as to permit the exposure of fresh cutting surfaces.

44. The instrument of claim 43, wherein the blade is rotatably mounted in grooves formed on the inside wall of said instrument.

45. The instrument of claim 3, wherein the handle comprises an enlarged proximal end for accommodating a storage compartment and for providing a grip.

46. The instrument of claim 18, wherein the handle comprises an enlarged proximal end for accommodating a storage compartment and for providing a grip.

47. The instrument of claim 24, wherein the handle comprises an enlarged proximal end for accommodating a storage compartment and for providing a grip.

48. The instrument of claim 27, wherein the handle comprises an enlarged proximal end for accommodating a storage compartment and for providing a grip.

49. The instrument of claim 24, wherein the blade comprises a blade of ring-like shape rotatably mounted in the instrument so as to permit the exposure of fresh cutting surfaces.

50. The instrument of claim 49, wherein the blade is rotatably mounted in grooves formed on the inside wall of said instrument.

51. The instrument of claim 24, wherein the handle comprises an enlarged proximal end for accommodating graft material and for providing a grip.

52. The instrument of claim 27, wherein the blade comprises a blade of ring-like shape rotatably mounted in the instrument so as to permit the exposure of fresh cutting surfaces.

53. The instrument of claim 52, wherein the blade is rotatably mounted in grooves formed on the inside wall of said instrument.

54. The instrument of claim 5, wherein the blade and channel have a clearance fit so as to enable the free insertion and removal of blade assemblies while the channel is constricted at the distal end to transfer cutting forces directly from the handle to the blade.

55. The instrument of claim 18, wherein the blade is mounted in a channel or groove formed on the handle, and the blade and channel have a clearance fit so as to enable the free insertion and removal of blade assemblies while the channel is constricted at the distal end to transfer cutting forces directly from the handle to the blade.

56. The instrument of claim 24, wherein the blade is mounted in a channel or groove formed on the handle, and the blade and channel have a clearance fit so as to enable the free insertion and removal of blade assemblies while the channel is constricted at the distal end to transfer cutting forces directly from the handle to the blade.

57. The instrument of claim 27, wherein the blade is mounted in a channel or groove formed on the handle, and the blade and channel have a clearance fit so as to enable the free insertion and removal of blade assemblies while the channel is constricted at the distal end to transfer cutting forces directly from the handle to the blade.

58. The instrument of claim 8, wherein the blade comprises stainless steel or monocrystalline sapphire.

59. The instrument of claim 18, wherein the blade is supported on a blade carrier which in turn is supported on said body.
